(19) United States
(12) Patent Application Publication
(10) Pub. No.: US 2026/0354777 A1
(43) Pub. Date: Nov. 12, 2026

(54) SYSTEMS AND METHODS FOR MEDICAL IMAGING

(71) Applicant: SHANGHAI UNITED IMAGING HEALTHCARE CO., LTD., Shanghai (CN)

(72) Inventors: Tiantian LI, Houston, TX (US); Dongxu YANG, Houston, TX (US); Xishan SUN, Houston, TX (US); Hongdi LI, Houston, TX (US)

(73) Assignee: SHANGHAI UNITED IMAGING HEALTHCARE CO., LTD., Shanghai (CN)

(21) Appl. No.: 19/671,191

(22) Filed: May 7, 2026

(30) Foreign Application Priority Data
May 7, 2025 (CN) 202510588074.X

(51) Int. Cl. A61B 6/06; A61B 6/00; A61B 6/03; G01T 1/164

(52) U.S. Cl. CPC A61B 6/06; A61B 6/037; A61B 6/4435; G01T 1/1642

(57) ABSTRACT

An imaging system is provided. The imaging system comprises an imaging device, wherein: the imaging device comprises a plurality of detector rings and a plurality of collimator rings, the plurality of detector rings are arranged side by side along an axial direction of the imaging device, each of the plurality of detector rings comprises a plurality of detection modules arranged at intervals along a circumferential direction of the imaging device, and the detection modules of adjacent detector rings are staggered along the circumferential direction, and each of the plurality of collimator rings is nested within a channel formed by one of the plurality of detector rings.

### CROSS-REFERENCE TO RELATED APPLICATIONS

This application claims priority of Chinese Patent Application No. 202510588074.X filed on May 7, 2025, the contents of which are incorporated herein by reference.

### TECHNICAL FIELD

The present disclosure generally relates to the field of medical imaging, and in particular to a Single photon emission computed tomography SPECT system.

### BACKGROUND

SPECT is a functional nuclear medicine imaging technique. The functional nuclear medicine imaging technique reconstructs distribution images of radiopharmaceuticals by detecting gamma photons released from radioactive nuclide decay within a body. In a SPECT system, a collimator is a key component located in front of a detector. The collimator performs spatial positioning and directional filtering of incident gamma photons through a specific channel structure. The specific channel structure ensures that only photons from specific directions can be received by the detector to form projection data. Among different types of collimators, a pinhole collimator can achieve high-resolution imaging due to an image magnification capability of the pinhole collimator. However, inherent physical properties of the pinhole collimator often result in low system sensitivity. A trade-off relationship exists between an imaging field of view (FOV) and detection efficiency.

In existing technologies, to meet the requirements of different imaging tasks for FOVs, a SPECT system based on pinhole collimators typically switches FOVs by replacing an entire collimator. However, a collimator needs to be made of high-density metals such as lead or tungsten to effectively shield radiation. The high-density metals cause the collimator to be bulky and heavy. The bulkiness and heaviness of the collimator require complex mechanical assistance for a replacement process. The replacement process is cumbersome and time-consuming. The replacement process cannot be performed online during a scan. The limitations severely restrict the clinical practicality and flexibility of a system. Furthermore, in a traditional full-ring SPECT system, data acquisition angles are limited by a number of flat-panel detectors arranged circumferentially and a fixed ring diameter. Limited acquisition angles can easily cause artifacts in image reconstruction due to information loss. The limited acquisition angles also reduce the spatial resolution of the SPECT system, which makes it difficult to meet requirements for the precise localization of small lesions. Simultaneously, the utilization efficiency of detector resources is relatively low, and the low utilization efficiency limits further improvement of system sensitivity.

Therefore, an imaging system, which can flexibly acquire imaging data from multiple angles through an optimized design of a collimator, is needed to improve the imaging sensitivity and resolution.

### SUMMARY

An imaging system is provided. The imaging system comprises an imaging device, wherein: the imaging device comprises a plurality of detector rings and a plurality of collimator rings, the plurality of detector rings are arranged side by side along an axial direction of the imaging device, each of the plurality of detector rings comprises a plurality of detection modules arranged at intervals along a circumferential direction of the imaging device, and the detection modules of adjacent detector rings are staggered along the circumferential direction, and each of the plurality of collimator rings is nested within a channel formed by one of the plurality of detector rings.

In some embodiments, for each of the plurality of collimator rings, the collimator ring is rotatable relative to the corresponding detector ring about the axial direction of the imaging device.

In some embodiments, for each of the plurality of collimator rings, the collimator ring comprises collimation units of a plurality of types, different types of collimation units having different structures, a rotation of the collimator ring causes one type of the collimation units to be in an active state, and one or more other types of the collimation units to be in an inactive state.

In some embodiments, the structures of the collimation units in the active state among the plurality of collimator rings are different.

In some embodiments, the structures of the collimation units in the active state among the plurality of collimator rings are the same.

In some embodiments, for each of the plurality of collimator rings, the collimation units comprise first collimation units of a first type and second collimation units of a second type, a first field of view (FOV) is achieved when the rotation of the collimator ring causes the first collimation units to be in the active state, a second FOV is achieved when the rotation of the collimator ring causes the second collimation units to be in the active state, and the first FOV is larger than the second FOV.

In some embodiments, the imaging system further comprises a processing device configured for: acquiring imaging data collected by the imaging device in at least one scan of a target subject, the imaging data comprising first imaging data corresponding to the first collimation units and second imaging data corresponding to the second collimation units; and generating a target image of the target subject based on the first imaging data and the second imaging data.

In some embodiments, the generating the target image of the target subject based on the first imaging data and the second imaging data comprises: reconstructing the first imaging data to obtain an initial image; and iteratively updating the initial image based on the second imaging data to generate the target image.

In some embodiments, the at least one scan comprises a first scan and a second scan, the first collimation units of each of the plurality of collimator rings are in the active state during the first scan, and the second collimation units of each of the plurality of collimator rings are in the active state during the second scan, a first duration of the first scan is shorter than a second duration of the second scan, and the target image is generated by performing a joint reconstruction on the first imaging data and the second imaging data based on the first duration and the second duration.

In some embodiments, the imaging system further comprises a processing device configured for: determining, based on information relating to a target subject to be scanned, the structure of the collimation units that are in the active state in each of the plurality of collimator rings.

In some embodiments, the plurality of detector rings include a target detector ring, the plurality of collimator rings include a target collimator ring, and the target collimator ring is nested within a channel formed by the target detector ring, the target collimator ring includes third collimation units and fourth collimation units, first projections of the third collimation units along a radial direction of the imaging device cover at least a portion of the plurality of detection modules of the target detector ring and are independent from each other, and second projections of the fourth collimation units along the radial direction of the imaging device cover at least a portion of the plurality of detection modules of the target detector ring and at least partially overlap with each other.

In some embodiments, the plurality of detection modules of the target detector ring include first detection modules and second detection modules, the first projections of the third collimation units along the radial direction of the imaging device cover the first detection modules and are independent from each other, the second projections of the fourth collimation units along the radial direction of the imaging device cover the second detection modules and at least partially overlap with each other.

In some embodiments, a size of the first detection modules is greater than or equal to a size of the second detection modules.

In some embodiments, a number of the first detection modules is greater than or equal to a number of the second detection modules.

In some embodiments, for each of the fourth collimation units, an area of an overlapping region of its corresponding second projection with second projections of other fourth collimation units accounts for no more than 50% of a total area of the second projection.

In some embodiments, wherein the target collimator ring includes first collimation modules and second collimation modules, the third collimation units being disposed on the first collimation modules, and the fourth collimation units being disposed on the second collimation modules; a size of the first collimation modules is greater than a size of the second collimation modules, and the first collimation modules and the second collimation modules are at a first distance from a central axis of the imaging device.

In some embodiments, the target collimator ring further includes third collimation modules, the third collimation modules being at a second distance from the central axis of the imaging device, the second distance being greater than the first distance, fifth collimation units are disposed on the third collimation modules, the fifth collimation units correspond to a first FOV, and the third collimation units and the fourth collimation units correspond to a second FOV, the first FOV being greater than the second FOV, the target collimator ring is rotatable relative to the target detector ring about the central axis of the imaging device to switch the target detector ring between the first FOV and the second FOV.

An imaging method is provided. The imaging method comprises: acquiring imaging data collected by the imaging device of claim 6 in at least one scan of a target subject, the imaging data including first imaging data corresponding to the first collimation units and second imaging data corresponding to the second collimation units; and generating a target image of the target subject based on the first imaging data and the second imaging data.

In some embodiments, the generating a target image of the target subject based on the first imaging data and the second imaging data comprises: reconstructing the first imaging data to obtain an initial image; and iteratively updating the initial image based on the second imaging data to generate the target image.

In some embodiments, the at least one scan comprises a first scan and a second scan, the first collimation units of each of the plurality of collimator rings being in the active state during the first scan, and the second collimation units of each of the plurality of collimator rings being in the active state during the second scan, a first duration of the first scan is shorter than a second duration of the second scan, and the target image is generated by performing a joint reconstruction on the first imaging data and the second imaging data based on the first duration and the second duration.

An imaging system is provided. The imaging system comprises an imaging device, wherein: the imaging device includes a detector and a collimator, the collimator being nested within a channel formed by the detector, the detector includes a plurality of detection modules spaced apart along a circumferential direction of the imaging device, and the collimator includes third collimation units and fourth collimation units, first projections of the third collimation units along a radial direction of the imaging device cover at least a portion of the detection modules and are independent from each other, second projections of the fourth collimation units along the radial direction of the imaging device cover at least a portion of the detection modules and at least partially overlap with each other.

In some embodiments, the plurality of detection modules include first detection modules and second detection modules, the first projections of the third collimation units along the radial direction cover the first detection modules and are independent from each other, and the second projections of the fourth collimation units along the radial direction cover the second detection modules and at least partially overlapping with each other.

In some embodiments, a size of the first detection modules is greater than or equal to a size of the second detection modules.

In some embodiments, a number of the first detection modules is greater than or equal to a number of the second detection modules.

In some embodiments, for each of the fourth collimation units, an area of an overlapping region of its corresponding second projection with second projections of other fourth collimation units accounts for no more than 50% of a total area of the second projection.

In some embodiments, the collimator includes first collimation modules and second collimation modules, the third collimation units being disposed on the first collimation modules, and the fourth collimation units being disposed on the second collimation modules; a size of the first collimation modules is greater than a size of the second collimation modules, and the first collimation modules and the second collimation modules are at a first distance from a central axis of the imaging device.

In some embodiments, the detector is a detector ring, the collimator is a collimator ring, the collimator ring further includes third collimation modules, the third collimation modules being at a second distance from the central axis of the imaging device, the second distance being greater than the first distance, fifth collimation units are disposed on the third collimation modules, the fifth collimation units correspond to a first FOV, and the third collimation units and the fourth collimation units correspond to a second FOV, the first FOV being greater than the second FOV, the collimator ring is rotatable relative to the detector ring about the central axis of the imaging device to switch the detector ring between the first FOV and the second FOV.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in an exemplary manner through embodiments, which are described in detail with reference to the accompanying drawings. These embodiments are not limiting. In these embodiments, the same reference numerals denote the same structures, wherein:

FIG. 1 is an exemplary schematic diagram of an imaging system according to some embodiments of the present disclosure;

FIG. 2 is an exemplary schematic diagram of an imaging device according to some embodiments of the present disclosure;

FIG. 3A is an exemplary top view of a detector ring according to some embodiments of the present disclosure;

FIG. 3B is an exemplary schematic diagram of an unfolded view of a detector ring along a circumferential direction according to some embodiments of the present disclosure;

FIG. 4A, FIG. 4B, and FIG. 4C are exemplary schematic diagrams of collimator rings according to some embodiments of the present disclosure;

FIG. 5 is an exemplary schematic diagram of a detection module receiving radiation rays according to some embodiments of the present disclosure;

FIG. 6 is an exemplary schematic diagram of a ray reception situation according to some embodiments of the present disclosure;

FIG. 7A and FIG. 7B are exemplary schematic diagrams of ray reception situations according to some embodiments of the present disclosure;

FIG. 8A and FIG. 8B are exemplary schematic diagrams of a first field of view (FOV) corresponding to first collimation units and a second FOV corresponding to second collimation units according to some embodiments of the present disclosure;

FIG. 9A, FIG. 9B, FIG. 9C, and FIG. 9D are exemplary schematic diagrams of an imaging device according to some embodiments of the present disclosure;

FIG. 10A and FIG. 10B are exemplary schematic diagrams of first projections and second projections according to some embodiments of the present disclosure;

FIG. 11A and FIG. 11B are exemplary schematic diagrams of target images according to some embodiments of the present disclosure;

FIG. 12A and FIG. 12B are exemplary schematic diagrams of an imaging device according to some embodiments of the present disclosure;

FIG. 13A and FIG. 13B are schematic diagrams of two states of an imaging device according to some embodiments of the present disclosure;

FIG. 14A and FIG. 14B are exemplary schematic diagrams of a first FOV and a second FOV according to some embodiments of the present disclosure;

FIG. 15 is an exemplary schematic diagram of an imaging device according to some embodiments of the present disclosure; and

FIG. 16 is a flowchart of a process for generating a target image of a target subject according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms ‘a,’ ‘an,’ and ‘the’ may be intended to include the plural forms as well, unless the context clearly indicates otherwise. It will be further understood that the terms ‘comprise,’ ‘comprises,’ and/or ‘comprising,’ ‘include,’ ‘includes,’ and/or ‘including,’ when used in this specification, specify the presence of stated features, integers, steps, operations, elements, and/or components, but do not preclude the presence or addition of one or more other features, integers, steps, operations, elements, components, and/or groups thereof.

It will be understood that the term ‘system,’′engine,′ ‘unit,’ ‘module,’ and/or ‘block’ used herein is one method to distinguish different components, elements, parts, sections or assemblies of different levels in ascending order. However, the terms may be displaced by another expression if they achieve the same purpose.

It will be understood that when a unit, engine, module, or block is referred to as being ‘on,’ ‘connected to,’ or ‘coupled to,’ another unit, engine, module, or block, it may be directly on, connected or coupled to, or communicate with the other unit, engine, module, or block, or an intervening unit, engine, module, or block may be present, unless the context clearly indicates otherwise. As used herein, the term ‘and/or’ includes any and all combinations of one or more of the associated listed items.

It will be understood that, although the terms ‘first,’ ‘second,’ ‘third,’ ‘fourth,’ etc., may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention.

Spatial and functional relationships between elements (for example, between crystal elements) are described using various terms, including ‘connected,’′engaged,′ ‘interfaced,’ and ‘coupled.’ Unless explicitly described as being ‘direct,’ when a relationship between first and second elements is described in the present disclosure, that relationship includes a direct relationship where no other intervening elements are present between the first and second elements, and also an indirect relationship where one or more intervening elements are present (either spatially or functionally) between the first and second elements. In contrast, when an element is referred to as being ‘directly’ connected, engaged, interfaced, or coupled to another element, there are no intervening elements present. Other words used to describe the relationship between elements should be interpreted in a like fashion (e.g., ‘between,’ versus ‘directly between,’ ‘adjacent,’ versus ‘directly adjacent,’ etc.). As used herein, the term ‘and/or’ includes any and all combinations of one or more of the associated listed items.

An anatomical structure shown in an image of a subject (e.g., a patient) may correspond to an actual anatomical structure existing in or on the subject's body. The term ‘object’ and ‘subject’ in the present disclosure are used interchangeably to refer to a biological object (e.g., a patient, an animal) or a non-biological object (e.g., a phantom). In some embodiments, the object may include a specific part, organ, and/or tissue of the object. For example, the object may include the head, the bladder, the brain, the neck, the torso, a shoulder, an arm, the thorax, the heart, the stomach, a blood vessel, soft tissue, a knee, a foot, or the like, or any combination thereof, of a patient.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

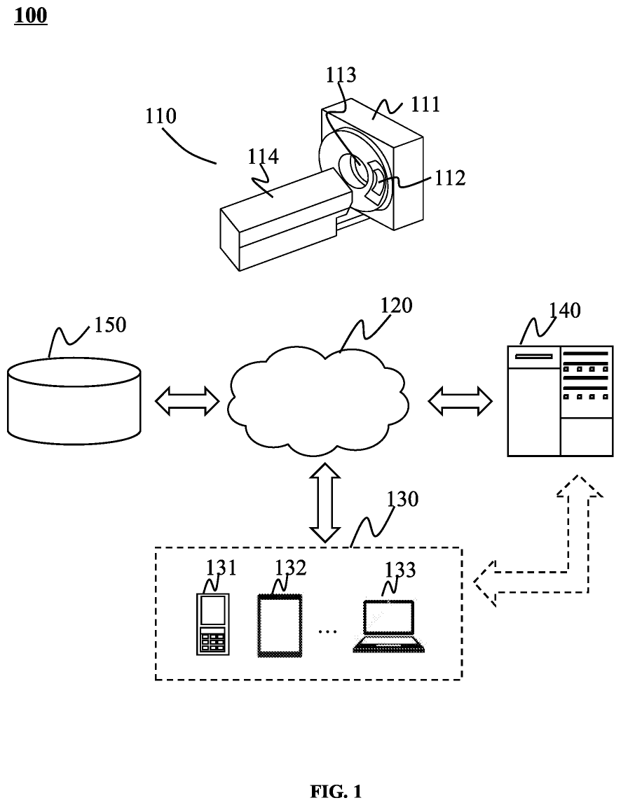

FIG. 1 is a schematic diagram illustrating an exemplary imaging system according to some embodiments of the present disclosure. As shown in FIG. 1, an imaging system 100 comprises an imaging device 110, a network 120, a terminal 130, a processing device 140, and a storage device 150.

The imaging device 110 can be used for scanning a target subject to acquire imaging data (for example, projection data, images). In some embodiments, the imaging device 110 may be a medical imaging device, for example, a SPECT (Single-Photon Emission Computed Tomography) imaging device, or other imaging devices, for example, a CT (Computed Tomography) imaging device, a PET (Positron Emission Tomography) imaging device, a SPECT-CT imaging device, a Single-Photon Emission Computed Tomography-Magnetic Resonance (SPECT-MR) imaging device, or the like.

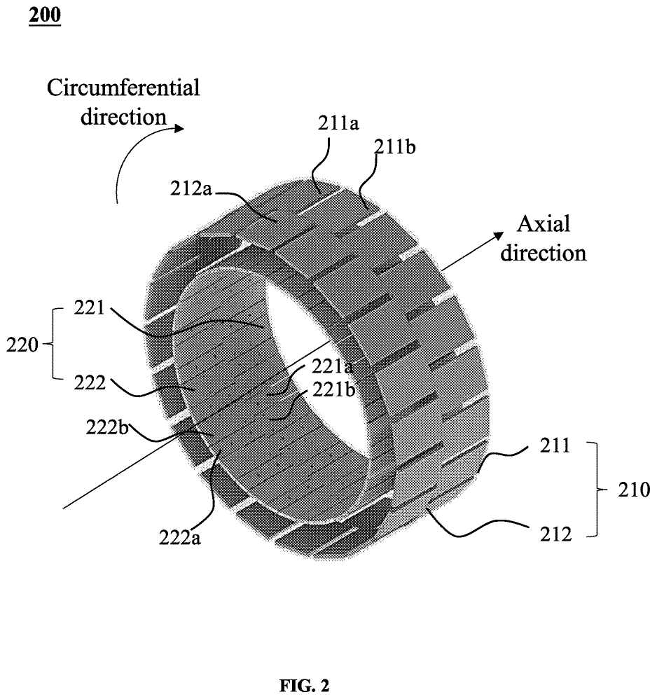

In some embodiments, the imaging device 110 may include a gantry 111, a detector 112, a scan region 113, and a scan bed 114. The target subject may be placed on the scan bed 114 and moved into the scan region 113 to be scanned. The gantry 111 can support the detector 112. In some embodiments, the detector 112 may include one or more detection modules. The detector 112 may include a single row of detection modules or a plurality of rows of detection modules. A detection module may include one or more detection units arranged along a circumferential direction of the gantry. In some embodiments, each of the plurality of detection units may be configured to generate an electrical signal in response to detecting radiation emitted by a subject located within a detection region enclosed by the plurality of detection units. Each of the plurality of detection units may be detachable. A detection unit may include a scintillator (for example, a cesium iodide detector), a semiconductor, pixels, or the like. In some embodiments, the imaging device 110 may further include a collimator (not shown in the figures), wherein the collimator includes a plurality of collimation units. More descriptions regarding the detector and the collimator may be found elsewhere in the present disclosure. See, e.g., FIGS. 2-15 and relevant descriptions thereof.

The network 120 includes any suitable network that can facilitate information and/or data exchange of the imaging device 110. In some embodiments, at least one component of the imaging system 100 (for example, the imaging device 110, the terminal 130, the processing device 140, the storage device 150) can exchange information and/or data with at least one other component in the imaging system 100 via the network 120. For example, the processing device 140 acquires imaging data from the imaging device 110 via the network 120. In some embodiments, the network 120 includes at least one network access point. For example, the network 120 includes wired and/or wireless network access points (for example, base stations and/or internet exchange points), and at least one component of the imaging system 100 can connect to the network 120 via the access points to exchange data and/or information.

The terminal 130 communicates and/or connects with the imaging device 110, the processing device 140, and/or the storage device 150. In some embodiments, the terminal 130 includes a mobile device 131, a tablet computer 132, a laptop computer 133, or the like, or any combination thereof. For example, the mobile device 131 includes a mobile control handle, a personal digital assistant (PDA), a smart phone, or the like, or any combination thereof. In some embodiments, the terminal 130 includes a display device, for example, a monitor. The display device can be used for displaying images obtained by imaging or other information, for example, medical images of a patient, a three-dimensional model, or an operation panel related to medical imaging, or the like. In some embodiments, the terminal 130 may be a portion of the processing device 140.

The processing device 140 can be used for processing data and/or information obtained from the imaging device 110, the terminal 130, the storage device 150, or other components of the imaging system 100. For example, the processing device 140 can be used for executing the imaging methods disclosed in some embodiments of the present disclosure. In some embodiments, the processing device 140 includes a single server or a server group. The server group may be centralized or distributed. In some embodiments, the processing device 140 may be local or remote. For example, the processing device 140 can access information and/or data from the imaging device 110, the storage device 150, and/or the terminal 130 via the network 120. In another example, the processing device 140 is directly connected to the imaging device 110, the terminal 130, and/or the storage device 150 to access information and/or data. In yet another example, the processing device 140 is installed on the imaging device 110. In some embodiments, the processing device 140 is implemented on a cloud platform. For example, the cloud platform includes a private cloud, a public cloud, a hybrid cloud, a community cloud, a distributed cloud, an inter-cloud, a multi-cloud, or the like, or any combination thereof.

The storage device 150 is used for storing data, instructions, and/or any other information. For example, the storage device 150 stores data obtained from the imaging device 110, the terminal 130, and/or the processing device 140. In some embodiments, the storage device 150 stores data and/or instructions used by the processing device 140 to execute or complete exemplary imaging methods described in the present disclosure. In some embodiments, the storage device 150 includes a mass memory, a removable memory, a volatile read-write memory, a read-only memory (ROM), or the like, or any combination thereof. In some embodiments, the storage device 150 is implemented on the cloud platform.

In some embodiments, the storage device 150 is connected to the network 120 to communicate with at least one other component in the imaging system 100 (for example, the processing device 140, the terminal 130). At least one component in the imaging system 100 can access data stored in the storage device 150 via the network 120. In some embodiments, the storage device 150 is a portion of the processing device 140. In some embodiments, the processing device 140 and the storage device 150 are integrated in the imaging device 110.

It should be noted that the above description is provided for illustrative purposes only and is not intended to limit the scope of the present disclosure. To a person of ordinary skill in the art, a plurality of changes and modifications may be made under the guidance of the content of the present disclosure. Features, structures, manners, and other features of the exemplary embodiments described in the present disclosure can be combined in various manners to obtain additional and/or alternative exemplary embodiments. For example, the storage device 150 is a data storage device including a cloud computing platform, for example, the public cloud, the private cloud, the community cloud, the hybrid cloud, or the like. However, these changes and modifications do not depart from the scope of the present disclosure.

FIG. 2 is a schematic diagram illustrating an imaging device 200 according to some embodiments of the present disclosure. The imaging device 200 comprises a plurality of detector rings 210 and a plurality of collimator rings 220. For example, as shown in FIG. 2, the imaging device 200 includes detector rings 211 and 212 and collimator rings 221 and 222. It should be understood that the imaging device 200 may include any number of detector rings or collimator rings. Furthermore, the imaging device 200 may include other components, such as the gantry, or the like.

A detector ring is a component in the imaging device configured to detect gamma rays emitted by radioactive isotopes. Radioactive tracers injected into the body of the target subject emit gamma rays when they decay. These gamma rays, after being captured by the detector ring, can be converted into electrical signals. The converted electrical signals, after a series of processing, can be used to generate an image of the target subject.

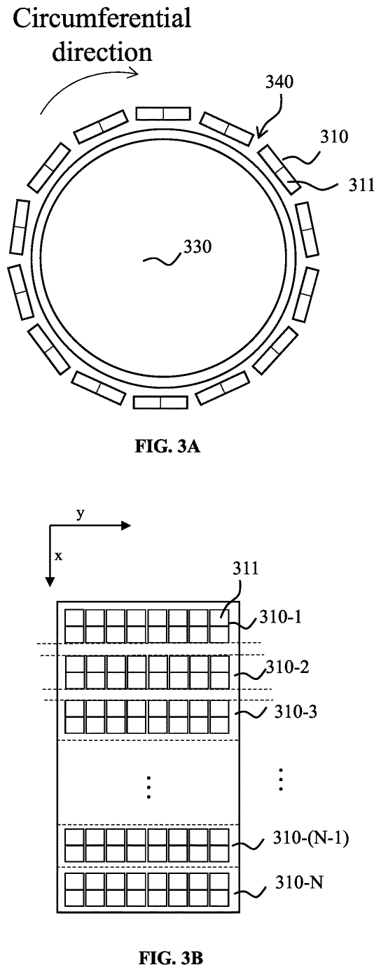

In some embodiments, each detector ring may include a plurality of detection modules arranged at intervals along the circumferential direction of the imaging device. A detection module may be a flat panel detector or a curved detector. The plurality of detection modules are arranged along the circumferential direction to form a tubular structure. Illustratively, as shown in FIG. 3A, FIG. 3A is an exemplary top view of a detector ring according to some embodiments of the present disclosure. In FIG. 3A, a plurality of detection modules 310 are arranged along the circumferential direction. Compared to arranging a single detection module, arranging the plurality of (two or more) detection modules along the circumferential direction can increase the effective detection area of the detector ring. For example, when the plurality of detection modules work simultaneously, they can acquire data from different angles, thereby improving resolution, enhancing sensitivity, and increasing redundancy and reliability. The circumferential direction refers to a direction along an edge of a circle, as indicated by the arrow in FIG. 3A. The axial direction of the imaging device refers to a direction along which the scanning bed moves when entering and leaving the scanning region. In some embodiments, the number of detection modules included in each detector ring may be set according to requirements, for example, it may be any number such as 20, 30, 40, 50, or the like. In some embodiments, the number of detection modules included in each detector ring is greater than or equal to 40.

The arrangement of the plurality of detection modules at intervals refers to that there is a certain gap between the adjacent detection modules in a detector ring. As shown in FIG. 3A, the space between the two adjacent detection modules in FIG. 3A is the gap, as indicated by arrow 340. FIG. 3B is an unfolded schematic diagram of a detector ring along the circumferential direction according to some embodiments of the present disclosure. The X direction in FIG. 3B corresponds to the circumferential direction in FIG. 3A, and the Y direction corresponds to the axial direction. The dashed line positions in FIG. 3B correspond to the gaps between the plurality of detection modules (310-1, 310-2, 310-3, . . . 310-(N−1), 310-N). In some embodiments, the gap between the adjacent detection modules may be replaced by one or more shielding modules. A shielding module refers to a component configured to prevent radiation rays from entering the detector modules, and may be made of high-density materials (for example, dense metal materials such as lead, tungsten, uranium, or the like).

In some embodiments, each detection module may include a plurality of detection units. For example, each detection module 310 may include a plurality of detection units 311. A detection unit is a basic unit of the detector ring. The detection unit refers to the smallest unit in the detector ring that can independently detect particles or radiation rays, and generate electrical signals. A plurality of detection units may be packaged to form a detection box, which is the detection module. The detection units in each detection module may be arranged in rows and columns forming an array. In some embodiments, each of the plurality of detection modules or the plurality of detection units may be detachable for efficiently adding, removing, and/or replacing the detection modules or the detection units from the imaging system.

In some embodiments, the plurality of detector rings are arranged along the axial direction of the imaging device. Merely by way of example, as shown in FIG. 2, the detector ring 211 and the detector ring 212 are arranged side by side along the axial direction of the imaging device. As another example, the plurality of detector rings are arranged at intervals along the axial direction of the imaging device. As yet another example, the plurality of detector rings are staggered along the axial direction of the imaging device (for example, the detector ring 211 is positioned upward, and the detector ring 212 is positioned downward).

In some embodiments, the detection modules of the adjacent detector rings are staggered. The staggered arrangement refers to that the detection modules of one detector ring and the detection modules of its adjacent detector ring are staggered along the circumferential direction, that is, the gaps between the detection modules of one detector ring are aligned with the detection modules of its adjacent detector ring. In this case, a projection of the detection modules of one detector ring along the axial direction will cover the gaps between the detection modules of its adjacent detector ring. For example, as shown in FIG. 2, the detection module 212a of the detector ring 212 is aligned with the gap between the detection modules 211a and 211b of the adjacent detector ring 211.

In some embodiments of the present disclosure, the imaging system introduces a design of the plurality of detector rings and the plurality of collimator rings. By staggering the detection modules of the adjacent detector rings, it can increase acquisition angles of imaging data, thereby improving image quality.

A collimator ring 220 is a component in an imaging device configured to constrain transmission paths of radiation rays. In some embodiments, a collimator ring may be a component made of materials with a large attenuation coefficient for radiation rays (for example, dense metal materials such as lead, tungsten, uranium, or the like) and containing a plurality of channels (e.g., parallel channels). The channels can enable the radiation rays to reach a detector ring along specific paths. In some embodiments, the shape of the channels may include circular, elliptical, polygonal shapes, or the like.

The plurality of detector rings 210 and the plurality of collimator rings 220 have a one-to-one correspondence, that is, each detector ring 210 has a corresponding collimator ring 220. Each collimator ring is nested within a channel (for example, an annular channel) formed by the corresponding detector ring. The channel formed by the detector ring refers to the region surrounded by the detection modules arranged along the circumferential direction. When performing detection on the target subject, the target subject may be located within the channel, so that the detection modules can detect radiation rays emitted from the body of the target subject. In some embodiments, the channel formed by the detector ring may be a cylinder-like three-dimensional space, wherein an axis of the cylinder penetrates through the center of the channel, and the plurality of detection modules are arranged around the axis to form an annular structure. As shown in FIG. 3A, a channel 330 is formed by the detector ring. As shown in FIG. 2, the collimator ring 221 is nested within the channel formed by the detector ring 211, and the collimator ring 222 is nested within the channel formed by the detector ring 212.

A collimator ring 220 may be formed by a plurality of collimation modules arranged along a circumferential direction. For example, referring to FIG. 2, the collimator ring 222 comprises collimation modules 222a and 222b, or the like, arranged along the circumferential direction. In some embodiments, no gap exists between the adjacent collimation modules. In some embodiments, a certain interval exists between the adjacent collimation modules. The arrangement of the collimation modules of a collimator ring is similar to that of the detection modules of a detector ring, and details thereof will not be repeated herein.

The collimation modules are provided with collimation units. A collimation unit is a basic unit of the collimator ring, and may independently allow radiation rays to pass through. In some embodiments, a collimation unit may be a small hole formed in the collimation module. The shape of the small hole may include circular, elliptical, polygonal, or the like. A collimation unit may be a divergent collimation unit and/or a convergent collimation unit.

In some embodiments, the collimator ring comprises collimation units of a plurality of types. The different types of collimation units have different structures. The structure of a collimation unit may be represented by structure parameters of the collimation unit. Typical structure parameters of the collimation unit include the aperture size (e.g., a diameter of a collimation unit), the aperture spacing (e.g., a distance between adjacent collimation units), the aperture length (e.g., a length of a collimation unit), the aperture angle (e.g., an angle of a collimation unit), a number of collimation units, a number of collimation unit columns, or the like.

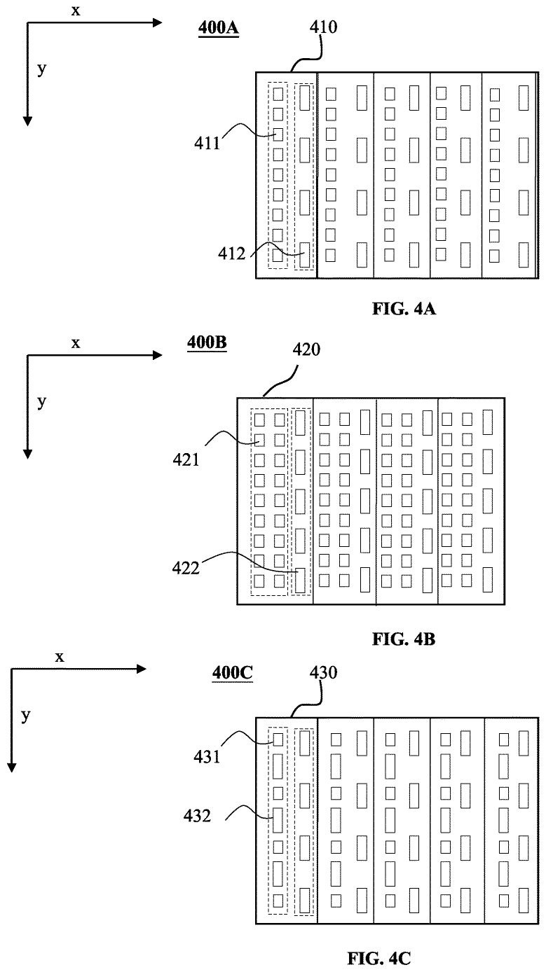

The different types of collimation units have at least one different structure parameter. For example, collimation units with different aperture sizes may be considered as different types of collimation units. FIGS. 4A, 4B, and 4C are schematic diagrams illustrating exemplary collimator rings according to some embodiments of the present disclosure. As shown in FIG. 4A, a collimator ring 400A comprises a plurality of collimation modules 410 arranged along a circumferential direction (i.e., an x-axis direction). A collimation module 410 comprises a column of collimation units 411 having a first aperture size and a column of collimation units 412 having a second aperture size. The second aperture size is greater than the first aperture size. As shown in FIG. 4B, a collimator ring 400B comprises a plurality of collimation modules 420 arranged along a circumferential direction (i.e., the x-axis direction). A collimation module 420 comprises two columns of collimation units 421 having the first aperture size and a column of collimation units 422 having the second aperture size.

In some embodiments, the collimation units of a plurality of types in a collimator ring are alternately arranged along the circumferential direction (x-direction). For example, as shown in FIG. 4A, along the circumferential direction, the arrangement order of the multiple types of collimation units may be collimation unit 411, collimation unit 412, collimation unit 411, collimation unit 412, and so on.

In some embodiments, a collimation module may be provided with a set of collimation units of the same type having the same structure parameters (for example, aperture size, aperture length, aperture angle, or the like) along an axial direction (y-direction) perpendicular to the circumferential direction. For example, as shown in FIG. 4A, a plurality of collimation units 411 are arranged side by side along the axial direction to form a column of small holes having the same structure parameters; a plurality of collimation units 412 are arranged side by side along the axial direction to form a column of large holes having the same structure parameters. As another example, as shown in FIG. 4B, a plurality of collimation units 421 are arranged side by side along the axial direction to form two columns of small holes having the same structure parameters; a plurality of collimation units 422 are arranged side by side along the axial direction to form a column of large holes having the same structure parameters. In some embodiments, a collimation module may be provided with different types of collimation units having different structure parameters (for example, aperture size, aperture length, aperture angle, or the like) along an axial direction (y-direction) perpendicular to the circumferential direction. For example, as shown in FIG. 4C, a collimator ring 400C comprises a plurality of collimation modules 430 arranged along a circumferential direction (i.e., the x-axis direction). A first column of collimation units on a collimation module 430 comprises collimation units 431 having the first aperture size and collimation units 432 having the second aperture size.

In some embodiments, different types of collimation units are disposed on the same collimation module. As shown in FIGS. 4A and 4B, collimation units of different aperture sizes are disposed on the same collimation module. In some embodiments, different types of collimation units are disposed on different collimation modules. As shown in FIG. 2, collimation units having a large aperture size are disposed on collimation modules 221a and 222a, and collimation units having a small aperture size are disposed on collimation modules 221b and 222b.

In some embodiments, for each of the plurality of collimator rings (or a portion thereof), the collimator ring is rotatable relative to the corresponding detector ring about the axial direction of the imaging device. Exemplarily, each collimator ring may be fixed on a rotor, and the rotor is driven by a motor via a belt to drive the collimator ring to rotate. In some embodiments, the collimator ring and its corresponding detector ring synchronously rotate about the axial direction. In some embodiments, the collimator ring rotates about the axial direction, while the detector ring is in a stationary state.

In some embodiments, when the collimator ring rotates about the axial direction, the collimation units of a plurality of types having different structures on the collimator ring also rotate about the axial direction, so as to change the positions of the plurality of types of collimation units along the circumferential direction. A rotation of the collimator ring causes one type of the collimation units to be in an active state, and one or more other types of the collimation units to be in the inactive state.

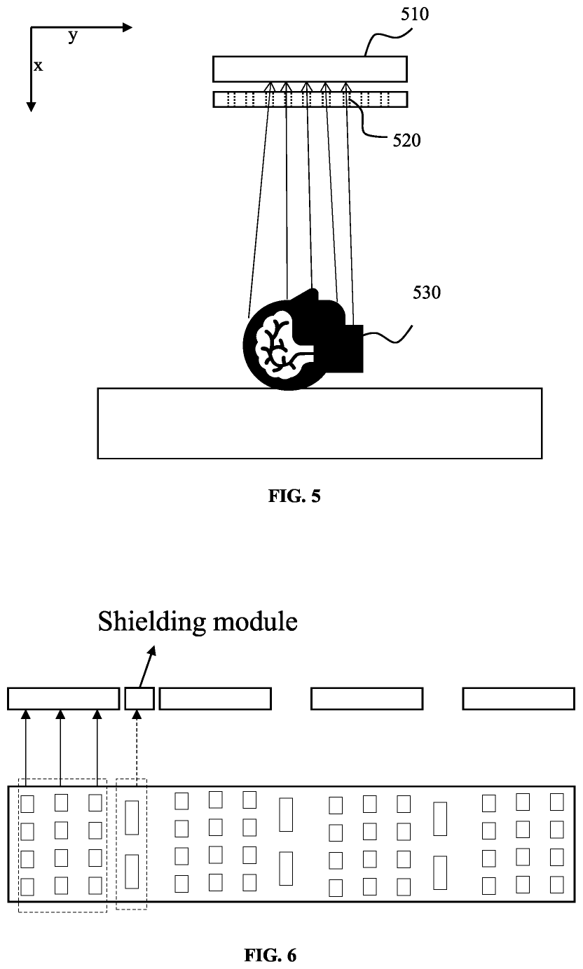

The active state of a collimation unit, which may also be referred to as a working state, refers to a state in which radiation rays passing through the collimation unit may be received by a detection module. As shown in FIG. 5, FIG. 5 is a schematic diagram illustrating an exemplary detection module receiving radiation rays according to some embodiments of the present disclosure. A target subject 530 is located in the channel formed by the detector ring. When radiation rays emitted from the body of the target subject can pass through a collimation unit 520 and reach a detection module 510, the collimation unit is considered to be in the active state.

The inactive state of the collimation unit, which may also be referred to as a non-working state, refers to a state in which radiation rays passing through the collimation unit cannot be received by a detection module. As previously described, a gap exists between two adjacent detection modules. When the collimation unit is in a position corresponding to the gap between the two adjacent detection modules, radiation rays passing through the collimation unit will not be able to reach a detection module, and thus cannot be received by any detection module. In this case, the collimation unit is in the inactive state.

In some embodiments, radiation rays passing through a collimation unit in the inactive state may irradiate a shielding module between the detection modules. As shown in FIG. 6, FIG. 6 is a schematic diagram illustrating an exemplary ray reception situation according to some embodiments of the present disclosure. The shielding module refers to a component configured to prevent radiation rays from entering a detector module. In some embodiments, the shielding module may be disposed in the gap between the adjacent detection modules. For example, the shielding module may be a component made of a high-density material (for example, lead or tungsten, or the like) disposed between the adjacent detection modules.

When the collimation unit is rotated along the circumferential direction of the channel formed by the detector ring to a position corresponding to the detection module, the radiation rays passing through the collimation unit will reach the detection module and can be received by the detection module. At this time, the collimation unit is in the active state. As described herein, when the position of the collimation unit and the position of the detection module are on the same radial direction, the position of the collimation unit is deemed as being corresponding to the position of the detection module. When the position of the collimation unit and the gap between the two adjacent detection modules are on the same radial direction, the position of the collimation unit is deemed as being corresponding to the position of the gap.

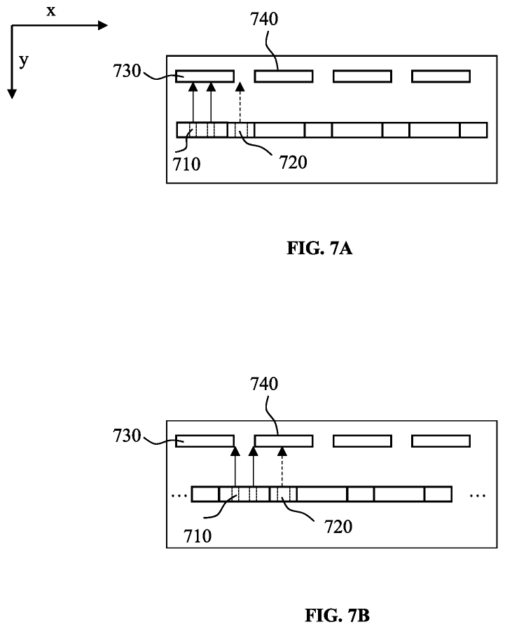

FIG. 7A and FIG. 7B are schematic diagrams illustrating exemplary ray reception situations according to some embodiments of the present disclosure. As shown in FIG. 7A, the collimator ring may include a collimation unit 710 and a collimation unit 720. When radiation rays passing through the collimation unit 710 can reach a detection module 730 and be received by the detection module 730, the collimation unit 710 is in the active state. When radiation rays passing through the collimation unit 720 reach a gap between two adjacent detection modules 730 and 740 and cannot be received by any detection modules, the collimation unit 720 is in the inactive state.

A rotation of the collimator ring along the circumferential direction may cause a relative change in the positions of the collimation units and the detection modules, thereby changing the active and inactive states of the collimation units. For example, the collimator ring in FIG. 7A, after being rotated along the circumferential direction (the x direction in FIG. 7A), is switched to the state as shown in FIG. 7B. At this time, the collimation unit 710 is aligned with the gap between the two adjacent detection modules 730 and 740. The radiation rays passing through the collimation unit 710 reach the gap between the two adjacent detection modules 730 and 740 and cannot be received by any detection modules. Thus, the collimation unit 710 is in the inactive state. The position of the collimation unit 720 is rotated to be aligned with the detection module 740. The radiation rays passing through the collimation unit 720 can reach the detection module 740 and be received by the detection module 740. Thus, the collimation unit 720 is in the active state.

In some embodiments of the present disclosure, each collimator ring comprises different types of collimation units. The collimator ring may be rotated to cause one type of the collimation units to be in the active state, thereby achieving a rapid and flexible switching of the collimation units to meet different scanning demands.

In some embodiments, the structures (or types) of the collimation units in the active state among the plurality of collimator rings are different. In the present disclosure, as long as the structures of the collimation units in the active state of at least two of the plurality of collimator rings are different, it can be considered that the structures of the collimation units in the active state among the plurality of collimator rings are different. For example, the collimation units in the active state of the collimator ring 221 are the single-row large-aperture pinholes in the collimation module 221a, and the collimation units in the active state of the collimator ring 222 are the double-row small-aperture pinholes in the collimation module 222b.

In some embodiments of the present disclosure, the structures of the collimation units in the active state among the plurality of collimator rings are different, so that the plurality of collimator rings can respectively obtain imaging data with complementary sensitivity/resolution characteristics. This increases the diversity of the acquired data, thereby improving the overall image quality.

In some embodiments, the structures (or types) of the collimation units in the active state among the plurality of collimator rings are the same. For example, the collimation units in the active state of the collimator ring 221 are the single-row large-aperture holes in the collimation module 221a, and the collimation units in the active state of the collimator ring 222 are the single-row large-aperture holes in the collimation module 222a.

In some embodiments of the present disclosure, the structures of the collimation units in the active state among the plurality of collimator rings are the same, thus the imaging data with the same sensitivity/resolution characteristic become sufficiently rich in count statistics. This reduces statistical noise and helps suppress artifacts that typically arise from insufficient counts at a given collection angle, resulting in a cleaner and more reliable reconstructed image.

In some embodiments, the imaging system 100 includes the processing device 140. The processing device 140 may be used for determining, based on information relating to a target subject to be scanned, the structure of the collimation units that is in the active state in each of the plurality of collimator rings. The information relating to the target subject may include a body part of the target subject, a size of the target subject, or the like. For example, if the target subject is a head, then the collimation units in the active state may be collimation units of a small aperture size to obtain an image with high resolution and a small FOV. As another example, if the target subject is a lung, then the collimation units in the active state may be collimation units of a large aperture size to obtain an image with low resolution and a large FOV. As yet another example, if the target subject is a region of interest (ROI) in an area (such as a heart in a chest), then the structures of the collimation units in the active state may simultaneously include collimation units of a small aperture size and collimation units of a large aperture size, so that a large FOV corresponding to the collimation units of the large aperture size covers the entire area, and a small FOV corresponding to the collimation units of the small aperture size covers the ROI, to obtain an image of the entire area and at the same time have a higher resolution for the ROI.

In some embodiments, the processing device 140 may first determine an FOV size based on the information relating to the target subject, and then determine the aperture size and the position of the collimation units based on the size and position of the detection modules and the FOV size. For example, after determining the FOV size to be achieved based on the information relating to the target subject, by drawing a tangent line of the FOV range based on the size and position of the detection modules, the aperture size and the position of the collimation units corresponding to the detection modules can be determined.

In some embodiments of the present disclosure, determining the structure of the collimation units in the active state in each of the plurality of collimator rings based on the information relating to the target subject to be scanned may enable the imaging data collected by the collimation units to be more suitable for the target subject, thereby improving the imaging quality of the imaging system.

Different types of collimation units with different structural parameters can meet different imaging requirements. For example, the aperture size, the aperture length, and the angle of the collimation units can all affect the FOV, the system sensitivity, and the spatial resolution of the collimation units. As described above, the configuration of the collimation units is defined by one or more structural parameters, such as the aperture size, the aperture length, the angle, or the like. By adopting different structural parameters, different sizes of FOVs can be obtained.

For example, an FOV range corresponding to a larger aperture size will be larger than an FOV corresponding to a smaller aperture size, because a large aperture can allow more radiation rays to pass through and can form a large FOV. At the same time, more radiation rays passing through the large aperture reach the detector module, which can improve sensitivity, while a small aperture can precisely limit a path range of the radiation rays passing through the collimator unit and improve spatial resolution. Therefore, if a larger FOV is needed, collimation units with a larger aperture size may be selected, and if a smaller FOV is needed, collimation units with a smaller aperture size may be selected.

In some embodiments, by setting an aperture size of the collimation units, different FOVs can be achieved. In some embodiments, the collimation units of the collimator ring include first collimation units of a first type and second collimation units of a second type. When the collimator ring rotates to cause the first collimation units to be in the active state, a first FOV is achieved. When the collimator ring rotates to cause the second collimation units to be in the active state, a second FOV is achieved. The first FOV is larger than the second FOV. For example, as shown in FIG. 2, when the collimator ring 221 rotates to cause the single-row first collimation units 221a with a large aperture size on the collimation module 221a to be in the active state, a larger first FOV is formed. When the collimator ring 222 rotates to cause the double-row second collimation units 222b with a small aperture size on the collimation module 222b to be in the active state, a smaller second FOV is formed.

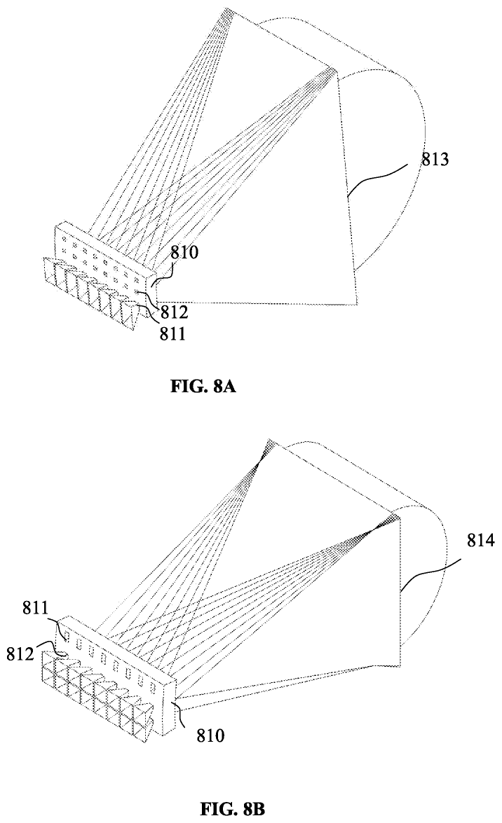

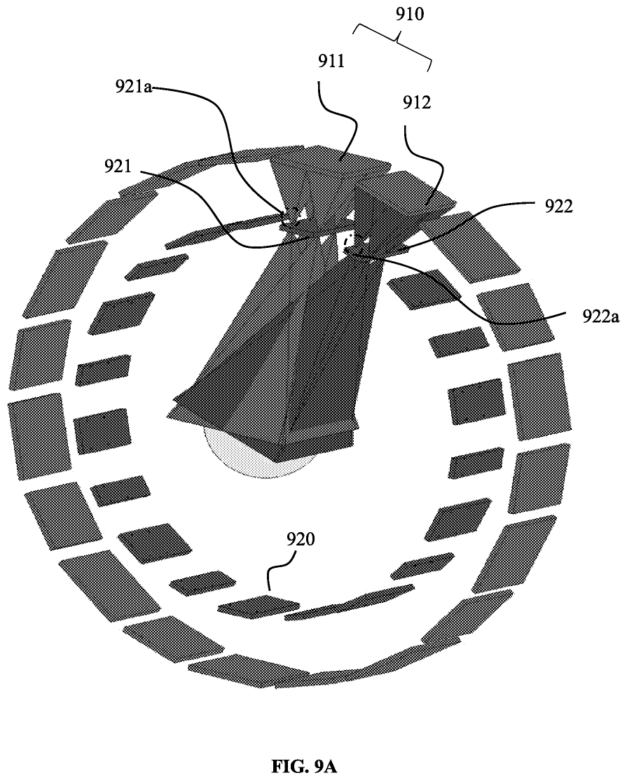

FIG. 8A and FIG. 8B are schematic diagrams illustrating an exemplary first FOV corresponding to the first collimation units and an exemplary second FOV corresponding to the second collimation units according to some embodiments of the present disclosure. For example, as shown in FIG. 8A and FIG. 8B, a collimation module 810 includes a single row of first collimation units 811 with a large aperture size and double rows of second collimation units 812 with a small aperture size. The first collimation units 811 in the active state in FIG. 8A form a first FOV 813, and the second collimation units 812 in the active state in FIG. 8B form a second FOV 814. The first FOV 813 is larger than the second FOV 814, and an aperture size of the first collimation units 811 corresponding to the first FOV 813 is larger than an aperture size of the second collimation units 812 corresponding to the second FOV 814.

In some embodiments, by setting a number of rows of the collimation units, different FOVs can also be obtained. For example, taking FIG. 8B as an example, increasing the number of rows of the second collimation units 812 on the collimation module 810 can increase a range of the second FOV.

In some embodiments, an FOV size may first be determined based on the information relating to the target subject (for example, a body part or a size of the target subject, or the like), and then the aperture size and the position may be determined based on the size and position of the detection modules and the FOV size.

In some embodiments, the imaging device includes a plurality of collimator rings and detector rings, the first collimation units of a portion of the plurality of collimator rings are in the active state, and the second collimation units of another portion of the plurality of collimator rings are in the active state. For example, as shown in FIG. 2, the first collimation units of the collimator ring 221 (such as the single-row large-aperture pinholes in the collimation module 221a) are in the active state, and the second collimation units of the collimator ring 222 (such as the double-row small-aperture pinholes in the collimation module 222b) are in the active state. At this time, the collimator ring 221 and the collimator ring 222 respectively achieve the first FOV and the second FOV.

In some embodiments, the first collimation units are in the active state in all the plurality of collimator rings. For example, the first collimation units of the collimator ring 221 (such as the single-row large-aperture pinholes in the collimation module 221a) are in the active state, and the first collimation units of the collimator ring 222 (such as the single-row large-aperture pinholes in the collimation module 222a) are in the active state. At this time, the collimator ring 221 and the collimator ring 222 both achieve the same first FOV.

In some embodiments, the second collimation units are in the active state in all the plurality of collimator rings. For example, the second collimation units of the collimator ring 221 (such as the double-row small-aperture pinholes in the collimation module 221b) are in the active state, and the second collimation units of the collimator ring 222 (such as the double-row small-aperture pinholes in the collimation module 222b) are in the active state. At this time, the collimator ring 221 and the collimator ring 222 both achieve the same second FOV.

In some embodiments of the present disclosure, collimation units of different structures correspond to different FOVs, which may enable imaging data of different resolutions and sensitivities to be collected based on the collimation units of different structures, thereby meeting different imaging requirements.

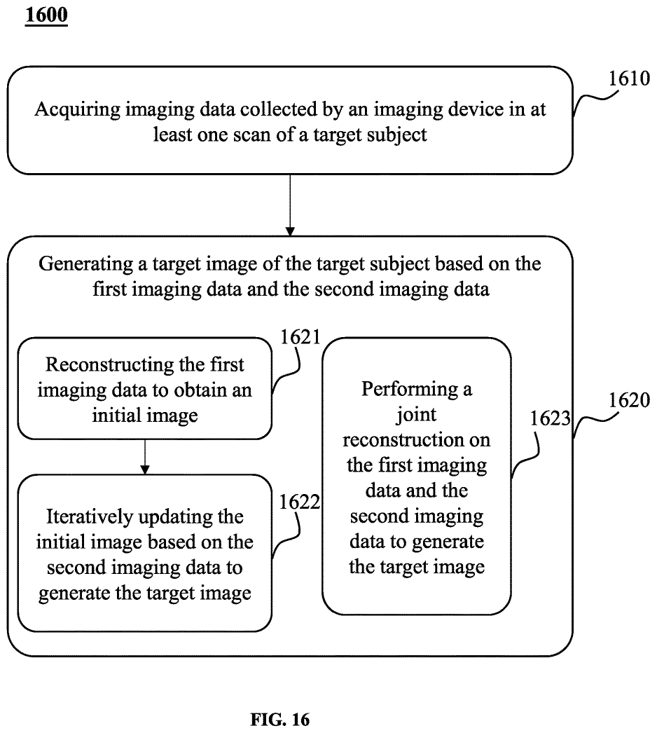

In some embodiments, the imaging system 100 includes the processing device 140. The processing device 140 is configured for acquiring imaging data collected by the imaging device in at least one scan of a target subject, and generating a target image of the target subject based on the imaging data. The imaging data includes first imaging data corresponding to the first collimation units and second imaging data corresponding to the second collimation units. Detailed descriptions regarding generating the target image of the target subject may refer to FIG. 16 and related descriptions thereof, which will not be repeated herein.

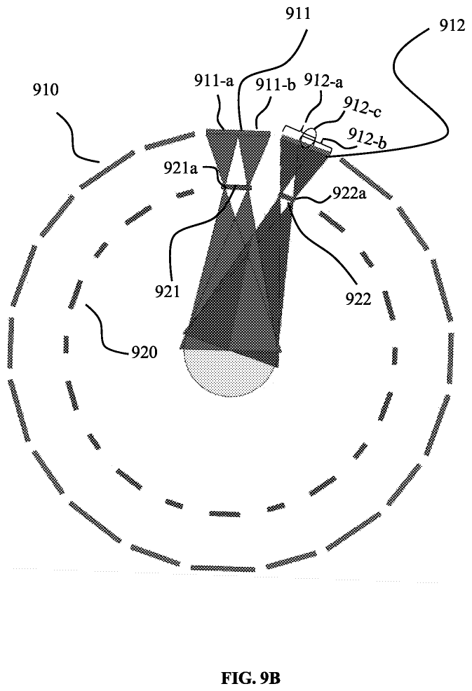

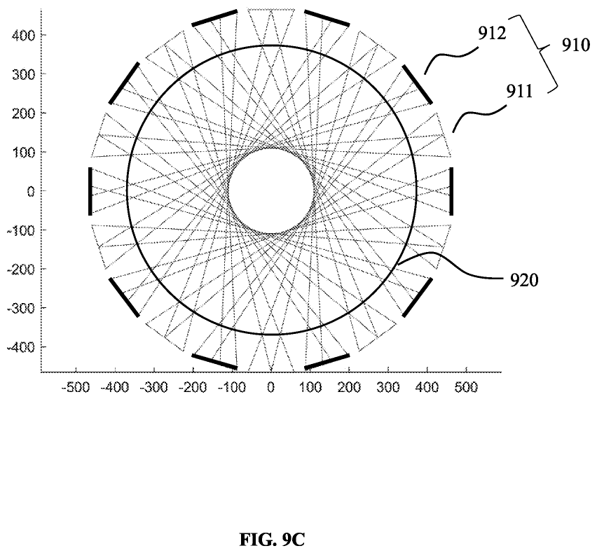

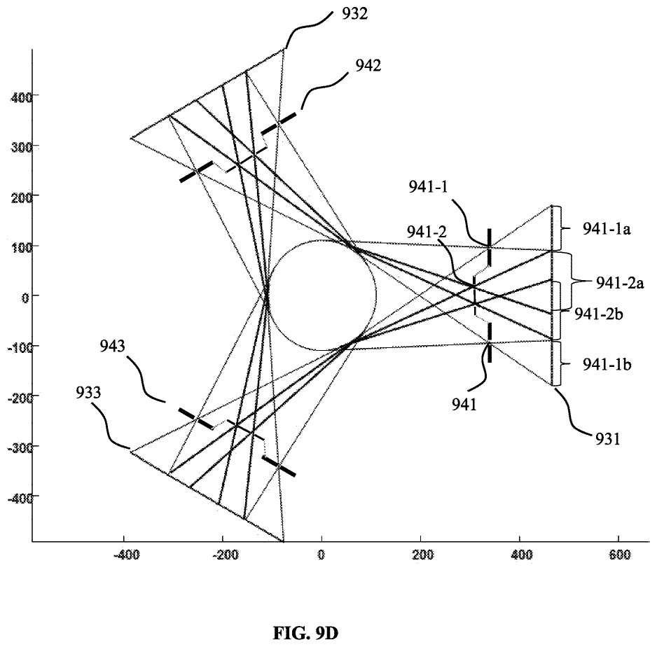

FIGS. 9A-9D are exemplary schematic diagrams of an imaging device according to some embodiments of the present disclosure. The imaging device includes a detector and a collimator, wherein the collimator is nested within a channel formed by the detector.

In some embodiments, as shown in FIGS. 9A-9C, the detector is a detector ring, and the collimator is a collimator ring, wherein the detector ring adopts the multiplexing design. The multiplexing design refers to reusing at least a portion of the detection modules, wherein the reused portion may simultaneously receive the radiation rays from a plurality of collimation units, so as to collect more data. In the present disclosure, a detector ring adopting the multiplexing design may also be referred to as a target detector ring, and a collimator ring corresponding to the target detector ring (i.e., the collimator ring nested within the channel formed by the target detector ring) may also be referred to as a target collimator ring. In some embodiments, when the imaging device includes a plurality of detector rings, at least one of the detector rings may adopt the multiplexing design (i.e., be a target detector ring).

For example, as shown in FIGS. 9A-9C, the imaging device includes a detector ring 910 (also referred to as a target detector ring 910) and a collimator ring 920 (also referred to as a target collimator ring 920). The detector ring 910 includes a plurality of detection modules spaced apart along a circumferential direction of the imaging device. The collimator ring 920 includes a plurality of collimation modules spaced apart along the circumferential direction of the imaging device. The collimator ring 920 is nested within a channel formed by the detector ring 910. The collimator ring 920 includes third collimation units 921a and fourth collimation units 922a. First projections of the third collimation units 921a along a radial direction of the imaging device cover at least a portion of the plurality of detection modules of the detector ring 910 and are independent from each other. Second projections of the fourth collimation units 922a along the radial direction of the imaging device cover at least a portion of the plurality of detection modules of the detector ring 910 and at least partially overlap with each other.

The first projections (or the second projections) refer to an image or a shape obtained by projecting radiation rays through the third collimation units (or the fourth collimation units) onto a plane where the corresponding detection modules are located. The plane where the detection modules are located refers to a plane passing through a geometric center of the detection modules and perpendicular to the radial direction. In some embodiments, if the detection modules are curved in shape, then the plane where the detection modules are located refers to a plane where a tangent plane at the geometric center of the detection modules is located. The first projections being independent from each other refers to that projections of radiation rays passing through a plurality of the third collimation units on the corresponding detection modules have no overlap. The second projections at least partially overlapping with each other refers to that projections of radiation rays passing through a plurality of the fourth collimation units on the corresponding detection modules have overlapping regions. When the second projections have overlapping regions, at least a portion of the detection modules may simultaneously receive the radiation rays passing through a plurality of the fourth collimation units, so as to achieve reuse of the at least a portion of the detection modules. By reusing at least a portion of the detection modules, the imaging data from more angles may be collected, thereby improving utilization efficiency of the detector and improving imaging quality.

In some embodiments, the plurality of detection modules of the detector ring 910 include first detection modules 911 and second detection modules 912, the first projections of the third collimation units 921a along the radial direction of the imaging device cover the first detection modules 911 and are independent from each other, and the second projections of the fourth collimation units 922a along the radial direction of the imaging device cover the second detection modules 912 and at least partially overlap with each other.

As shown in FIGS. 9A and 9B, a collimation module 921 (also referred to as a first collimation module) is provided with two rows of third collimation units 921a with larger row spacing, and the first projections 911-a and 911-b of the two rows of the third collimation units 921a along the radial direction of the imaging device cover the corresponding first detection module 911 and are independent from each other. A collimation module 922 (also referred to as a second collimation module) is provided with two rows of fourth collimation units 922a with smaller row spacing, and the second projections 912-a and 912-b of the two rows of the fourth collimation units 922a along the radial direction of the imaging device cover the corresponding second detection module 912 and at least partially overlap with each other. The second projections 912-a and 912-b have an overlapping region 912-c. In the embodiment of FIGS. 9A-9C, a distance between the third collimation units 921a and the central axis of the imaging device is equal to a distance between the fourth collimation units 922a and the central axis.

The first projections (or the second projections) may completely or partially cover their corresponding detection modules. For example, as shown in FIGS. 9A, 9B, and 9C, the first projections completely cover the first detection modules, and the second projections completely cover the second detection modules. As another example, the first projections and the second projections respectively cover a portion of the corresponding detection modules.

In some embodiments, as shown in FIGS. 9A and 9B, a size of the first detection modules 911 is greater than a size of the second detection modules 912. In some embodiments, as shown in FIG. 9C, a size of the first detection modules 911 is equal to a size of the second detection modules 912.

In some embodiments, a number of the third collimation units is greater than or equal to a number of the fourth collimation units. This may ensure that a number of independent projections is greater than a number of overlapping projections, so that the imaging data corresponding to the overlapping projection regions can be determined during subsequent image reconstruction.

In some embodiments, a number of the first detection modules 911 is equal to a number of the second detection modules 912. In this case, as shown in FIGS. 9A-9C, the first detection modules 911 and the second detection modules 912 are alternately arranged. In some embodiments, a number of the first detection modules 911 is greater than the second detection modules 912. In this case, one second detection module 912 may be arranged every plurality of the first detection modules 911. In some embodiments, a number of the second detection modules 912 is greater than a number of the first detection modules 911. In some embodiments, the detector may only include the second detection modules 912, which means all the detection modules on the detector adopt the multiplexing design.

In some embodiments, for each of the fourth collimation units, an area of an overlapping region (for example, the overlapping region 912-c in FIG. 9B) between its corresponding second projection and second projections of other fourth collimation units accounts for no more than a preset proportion of a total area of the second projection (for example, the second projection 912-a or 912-b in FIG. 9B). Merely by way of example, taking the fourth collimator unit 922a as an example, an area of the overlapping region 912-c accounts for no more than the preset proportion of the second projection 912-a. The preset proportion may be set manually. For example, the preset proportion may be any value between 50% to 70%. Thus, the data collected by detection modules corresponding to non-overlapping regions can provide sufficient information for determining the data collected by detection modules corresponding to the overlapping regions, so as to achieve the image reconstruction and avoid image artifacts.

In some embodiments, a collimation module may simultaneously be provided with one or more third collimation units and one or more fourth collimation units. Alternatively, a collimation module may only be provided with third collimation units, or only with fourth collimation units. For example, as shown in FIGS. 9A and 9B, the collimator ring 920 includes first collimation modules 921 and second collimation modules 922. A size of the first collimation modules 921 is greater than a size of the second collimation modules 922. The first collimation modules 921 may be provided with two rows of third collimation units 921a with larger row spacing, and the second collimation modules 922 may be provided with two rows of fourth collimation units 922a with smaller row spacing. The first collimation modules 921 and the second collimation modules 922 are at a first distance from the central axis of the imaging device.

In some embodiments, a detector and a collimator may be non-ring structures. For example, the detector comprises two or three detection modules uniformly arranged along the circumferential direction. Among the two or three detection modules, the same included angle exists between any two adjacent detection modules. These detection modules have the same size. For example, taking the detector comprising two detection modules as an example, the two detection modules may be placed in parallel around a target subject (i.e., there is an included angle of 180°). As another example, as shown in FIG. 9D, the detector comprises three detection modules 931, 932, and 933, and there is an included angle of 120° between any two adjacent detection modules among the three detection modules 931, 932, and 933. The collimator comprises three collimation modules 941, 942, and 943. The three collimation modules are nested within the channels formed by the three detection modules and correspond one-to-one to the three detection modules 931, 932, and 933.

Each collimation module may comprise a plurality of third collimation units and a plurality of fourth collimation units. Taking the collimation module 941 as an example, the collimation module 941 comprises a plurality of third collimation units 941-1 (represented by thick line segments in the figure) and a plurality of fourth collimation units 941-2 (represented by thin line segments in the figure). First projections 941-1a and 941-1b of the plurality of third collimation units 941-1 along a radial direction of the imaging device cover edge regions of the detection module 931 and are independent from each other. Second projections 941-2a and 941-2b of the plurality of fourth collimation units along the radial direction of the imaging device cover a middle region of the detection module 931 and at least partially overlap with each other. In the embodiment of FIG. 9D, a distance of the third collimation units 941-1 from the central axis of the imaging device is greater than a distance of the fourth collimation units 941-2 from the central axis.

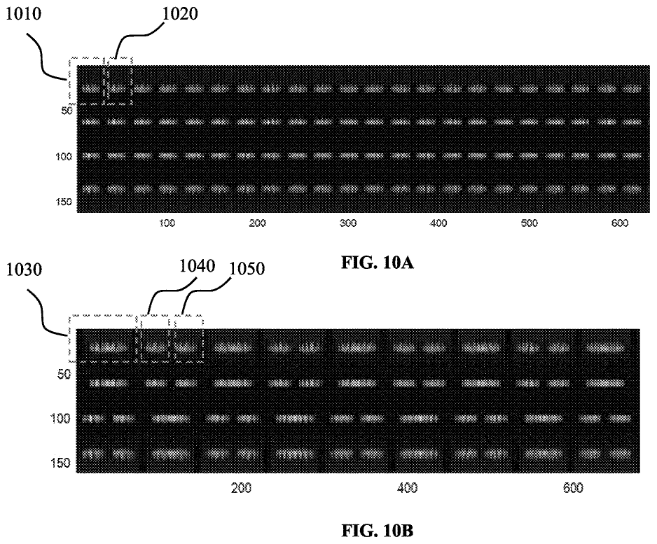

FIGS. 10A and 10B are exemplary schematic diagrams illustrating a first projection and a second projection according to some embodiments of the present disclosure. In the figures, each line segment represents a projection projected from a collimation unit onto a plane where the detection module is located. As shown in FIG. 10A, first projections (e.g., first projections 1010 and 1020) are independent from each other. As shown in FIG. 10B, some second projections overlap (e.g., 1030 is a total projection generated after projections corresponding to two collimation units overlap), and some other projections are independent from each other (e.g., projections 1040 and 1050). The independent first projection 1010 only comprises information of a single projection angle, while the overlapping second projection 1030 comprises information of different projection angles.

In some embodiments, the processing device 140 may reconstruct the imaging data collected by the detection modules to obtain a target image. A reconstruction algorithm may include but is not limited to Maximum Likelihood Expectation Maximization (MLEM), Least Squares (LS), or the like.

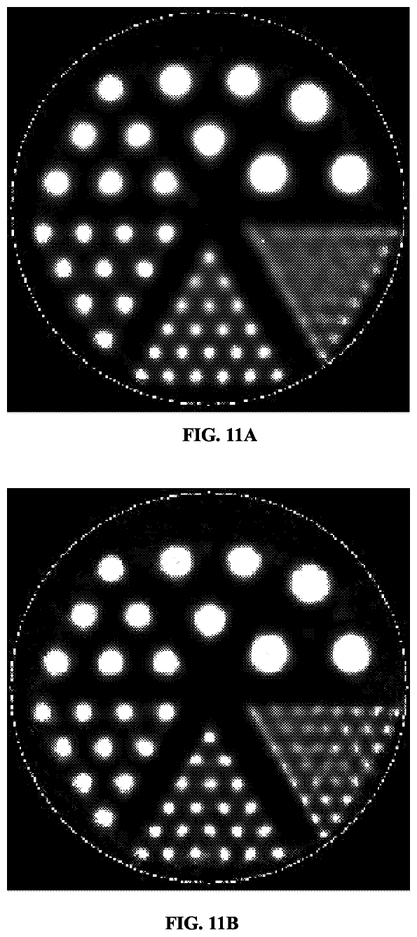

FIGS. 11A and 11B are exemplary schematic diagrams illustrating the target image according to some embodiments of the present disclosure. FIG. 11A is a target image obtained by reconstructing the imaging data collected only by a first detection module, and FIG. 11B is a target image obtained based on the imaging data collected by the first detection module and a second detector module. By comparing FIG. 11A and FIG. 11B, it can be seen that a portion of the imaging data collected by the second detector comprises information corresponding to different projection angles, which may thereby improve local clarity.

It should be noted that the terms ‘first’, ‘second’, ‘third’, and ‘fourth’ in the present disclosure are merely for the convenience of describing collimation units in different embodiments. A first or a second collimation unit may also be used as a third or a fourth collimation unit to form overlapping or non-overlapping projections. The third collimation units and the fourth collimation units may also be used as first or second collimation units to form different FOVs. In some embodiments, the third collimation units and the fourth collimation units may have different structures. Detailed descriptions of collimation units of a plurality of types with different structures may be referred to in FIGS. 2 to 8 and related descriptions thereof.

In some embodiments, the collimator ring 920 is rotatable relative to the detector ring 910 about the central axis of the imaging device, and a rotation causes one type of the collimation units to be in an active state and one or more other types of the collimation units to be in an inactive state. In some embodiments, by enabling the collimator ring 920 to rotate relative to the detector ring 910 about the central axis of the imaging device, a switching of the FOV may be achieved.

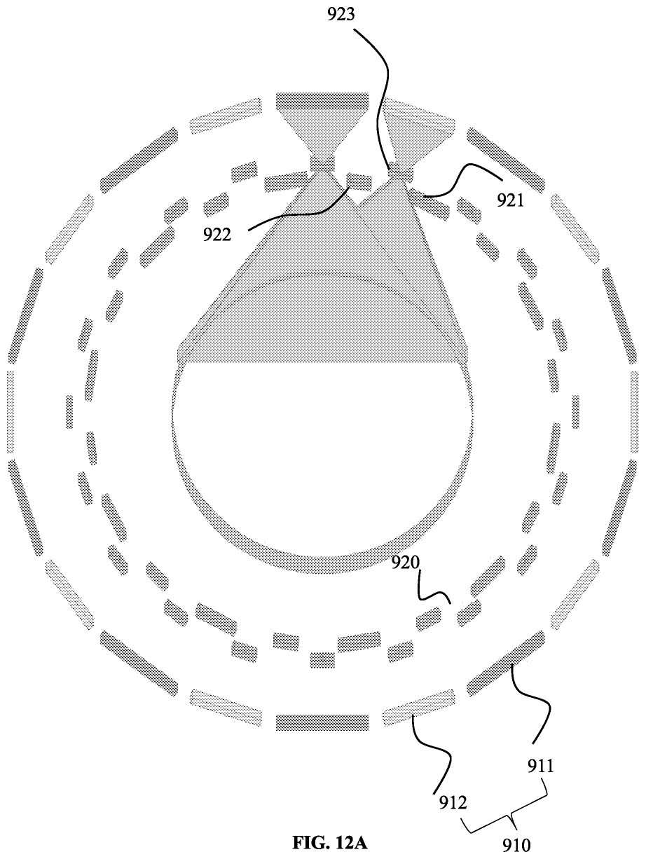

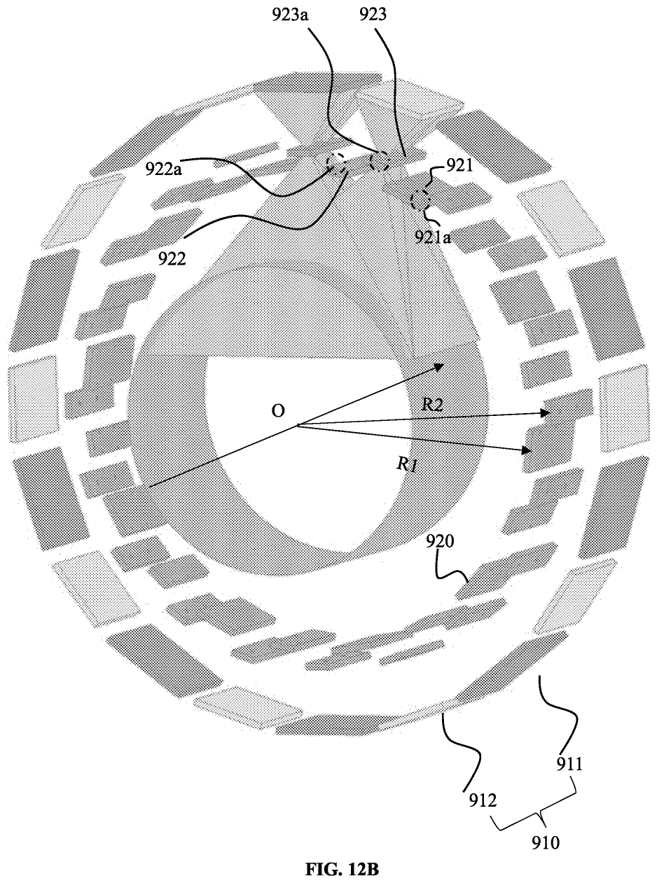

FIGS. 12A and 12B are exemplary schematic diagrams illustrating an imaging device according to some embodiments of the present disclosure. The imaging device shown in FIGS. 12A and 12B is similar to the imaging device shown in FIGS. 9A and 9B, except that the imaging device shown in FIGS. 12A and 12B further comprises a third collimation module 923. As shown in FIGS. 12A and 12B, the collimator ring 920 comprises the first collimation module 921, the second collimation module 922, and the third collimation module 923. The first collimation module 921 and the second collimation module 922 are at a first distance R1 from the central axis O of the imaging device, the third collimation module 923 is at a second distance R2 from the central axis O of the imaging device, the second distance R2 being greater than the first distance R1.

The third collimation units 921a are disposed on the first collimation module 921, the fourth collimation units 922a are disposed on the second collimation module 922, and fifth collimation units 923a are disposed on the third collimation module 923. The fifth collimation units 923a are single-row pinholes with a larger aperture, and can achieve a larger first FOV. The third collimation units 921a and the fourth collimation units 922a are double-row pinholes with a smaller aperture, and can achieve a smaller second FOV. In this case, the fifth collimation units 923a may serve as first collimation units for achieving a large FOV, and the third collimation units 921a and the fourth collimation units 922a may serve as second collimation units for achieving a small FOV.

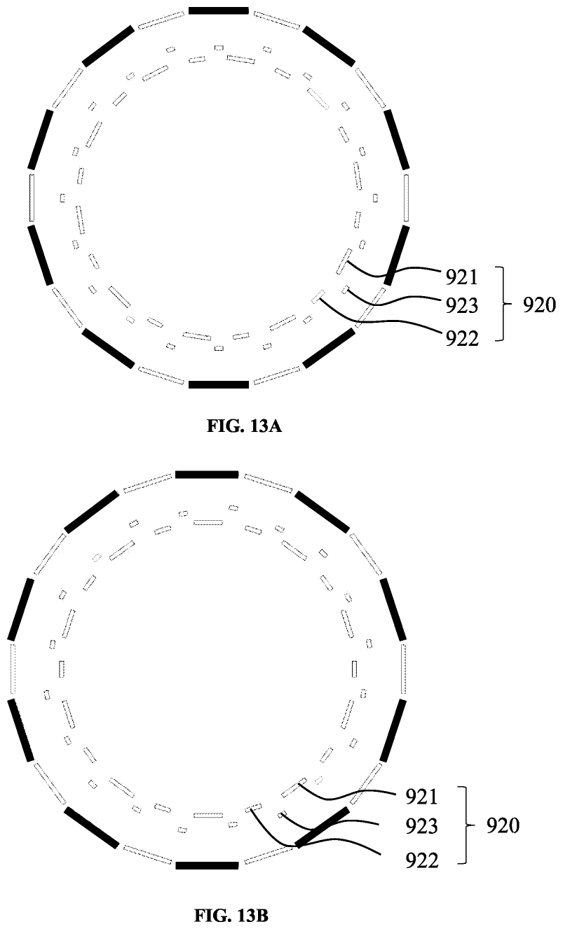

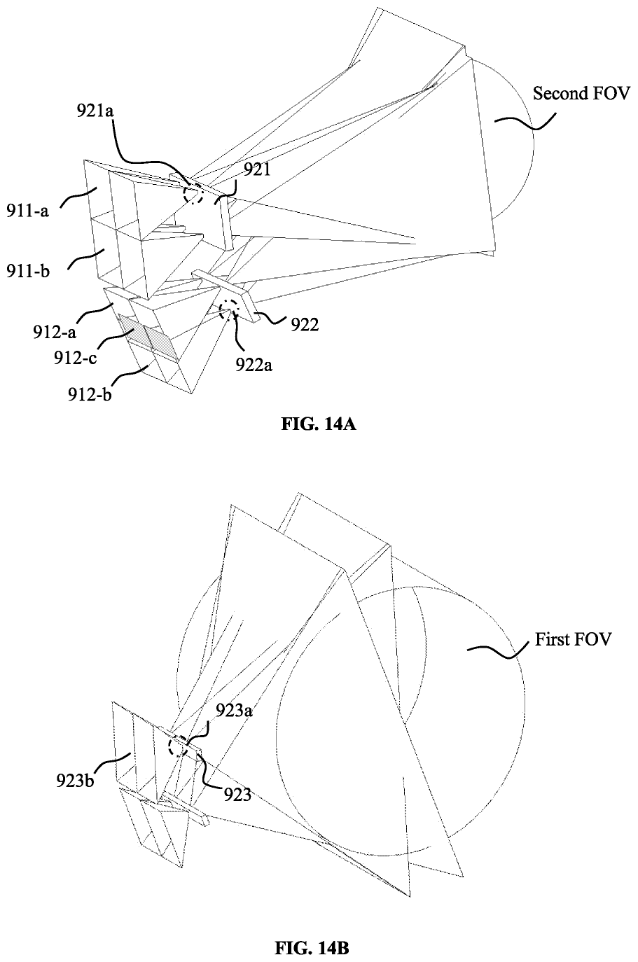

The collimator ring 920 is rotatable relative to the detector ring 910 about the central axis O (or the axial direction) of the imaging device to switch between the first FOV and the second FOV. For example, FIGS. 13A and 13B are exemplary schematic diagrams illustrating two states of an imaging device according to some embodiments of the present disclosure. FIGS. 14A and 14B are exemplary schematic diagrams illustrating a first FOV and a second FOV according to some embodiments of the present disclosure. The collimator ring 920 comprises the first collimation module 921 with the double-row third collimation units 921a (having the small aperture size), the second collimation module 922 with the double-row fourth collimation units 922a (having the small aperture size), and the third collimation module 923 with the single-row fifth collimation units 923a (having the large aperture size).

As shown in FIGS. 13A and 14B, when the fifth collimation units 923a on the third collimation module 923 are in an active state, and the third collimation units 921a on the first collimation module 921 and the fourth collimation units 922a on the second collimation module 922 are in an inactive state, the collimator ring 920 switches to the first FOV (i.e., a large field of view) corresponding to the fifth collimation units 923a. Projections 923b of the fifth collimation units 923a along the radial direction of the imaging device are independent from each other, and the detector ring does not adopt the multiplexing design.

As shown in FIGS. 13B and 14A, when the fifth collimation units 923a on the third collimation module 923 are in an inactive state, and the third collimation units 921a on the first collimation module 921 and the fourth collimation units 922a on the second collimation module 922 are in an active state, the collimator ring 920 switches to the second FOV (i.e., a small field of view) corresponding to the third collimation units 921a and the fourth collimation units 922a. In this case, the first projections 911-a and 911-b of the third collimation units 921a along the radial direction of the imaging device are independent from each other, and the second projections 912-a and 912-b of the fourth collimation units 922a along the radial direction of the imaging device have an overlapping region 912-c. In this case, the detector ring adopts the multiplexing design.

In some embodiments of the present disclosure, by disposing collimation units with different aperture sizes on different collimation modules, a switching of different FOVs may be easily achieved while implementing the multiplexing design.

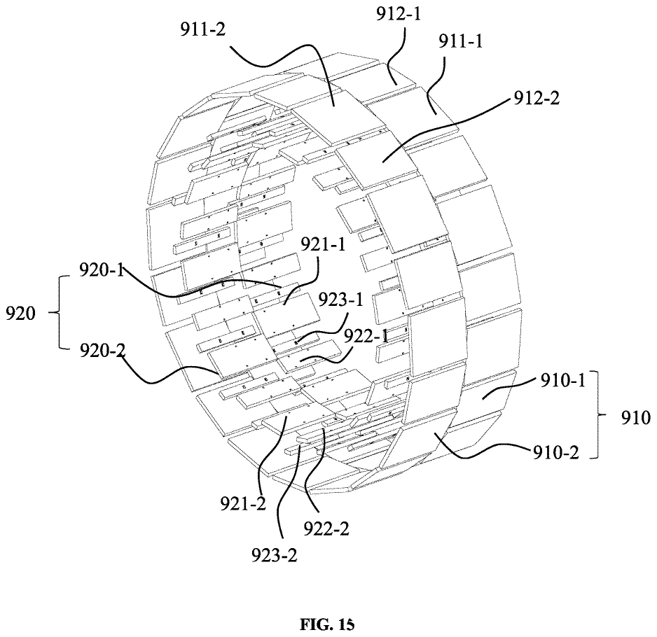

FIG. 15 is an exemplary schematic diagram of an imaging device according to some embodiments of the present disclosure. The imaging device includes a plurality of detector rings and a plurality of collimator rings. The plurality of detector rings are arranged side by side along an axial direction of the imaging device, and the detection modules of the adjacent detector rings are staggered.

As shown in FIG. 15, the imaging device includes detector rings 910-1 and 910-2, and collimator rings 920-1 and 920-2. Each detector ring may comprise a plurality of detection modules arranged at intervals along the circumferential direction of the imaging device. For example, the detector ring 910-1 may comprise first detection modules 911-1 and second detection modules 912-1, wherein a size of the first detection modules 911-1 is greater than a size of the second detection modules 912-1. A plurality of detection modules in the detector ring 910-2 may comprise first detection modules 911-2 and second detection modules 912-2, wherein a size of the first detection modules 911-2 is greater than a size of the second detection modules 912-2. Each of the first detection modules 911-2 and the second detection modules 912-2 of the detector ring 910-2 is aligned with a gap between the first detection modules 911-1 and the second detection modules 912-1 of the adjacent detector ring 910-1. Further descriptions regarding first detection modules and second detection modules may refer to FIG. 9A to FIG. 9C and related descriptions thereof.

Each collimator ring may comprise a plurality of collimation modules and collimation units. For example, the collimator ring 920-1 may comprise first collimation modules 921-1, second collimation modules 922-1, and third collimation modules 923-1. The first collimation modules 921-1 include four third collimation units of a small aperture size, the second collimation modules 922-1 include four fourth collimation units of a small aperture size, and the third collimation modules 923-1 include two fifth collimation units of a large aperture size. The first collimation modules 921-1 and the second collimation modules 922-1 are at an equal distance from the central axis of the imaging device. The third collimation modules 923-1 are at a relatively farther distance from the central axis of the imaging device.

The collimator ring 920-2 may comprise first collimation modules 921-2, second collimation modules 922-2, and third collimation modules 923-2. The first collimation modules 921-2 include four third collimation units of a small aperture size, the second collimation modules 922-2 include four fourth collimation units of a small aperture size, and the third collimation modules 923-2 include two fifth collimation units of a large aperture size. The first collimation modules 921-2 and the second collimation modules 922-2 are at an equal distance from the central axis of the imaging device. The third collimation modules 923-2 are at a relatively farther distance from the central axis of the imaging device.

In some embodiments, the structures of the collimation units in the active state among at least two collimator rings are different. For example, referring to FIG. 15, the collimation units in the active state in the collimator ring 920-1 may be the fifth collimation units, and the collimation units in the active state in the collimator ring 920-2 may be the third collimation units and the fourth collimation units. In this case, a situation of the collimator ring 920-1 and the detector ring 910-1 is similar to that of FIG. 13A and FIG. 14B. The fifth collimation units may serve as the first collimation units to form the first FOV, and the detector ring 910-1 has no multiplexing design. A situation of the collimator ring 920-2 and the detector ring 910-2 is similar to that of FIG. 13B and FIG. 14A. The third collimation units and the fourth collimation units may serve as the second collimation units to form the second FOV, and the detector ring 910-2 has the multiplexing design. In some other embodiments, the collimation units in the active state in the collimator ring 920-1 and the collimator ring 920-2 may all be the fifth collimation units, or may all be the third collimation units and the fourth collimation units.

In some embodiments, the first projections of a plurality of third collimation units along a radial direction of the imaging device cover first detection modules and are independent from each other, and the second projections of a plurality of fourth collimation units along a radial direction of the imaging device cover second detection modules and have at least partially overlapping regions. For example, referring to FIG. 15 and taking the collimator ring 920-1 as an example, the four pinholes with the small aperture size included in the larger first collimation modules 921-1 are the third collimation units. The first projections of the third collimation units along a radial direction of the imaging device cover the first detection modules 911-1 and are independent from each other. The four pinholes with the small aperture size included in the smaller second collimation modules 922-1 are the fourth collimation units. The second projections of the fourth collimation units along the radial direction of the imaging device cover the second detection modules 912-1 and have at least partially overlapping regions. Detailed descriptions regarding the first projections and the second projections may refer to FIG. 9A to FIG. 9C and related descriptions thereof.

It should be understood that the embodiments and related descriptions in FIGS. 2-15 are merely for illustrative purposes and are not intended to be limiting. These embodiments may be arbitrarily combined or adjusted.

FIG. 16 is a flowchart illustrating an exemplary process 1600 for generating a target image of a target subject according to some embodiments of the present disclosure. The process 1600 may be implemented as a set of instructions (e.g., an application) stored in a storage device (e.g., the storage device 150). The processing device 140 may execute the set of instructions and may accordingly be directed to perform the process 1600.

In 1610, the processing device 140 acquires imaging data collected by the imaging device in at least one scan of a target subject.

The imaging device may be any imaging device disclosed in the present disclosure, such as any of the imaging devices shown in FIG. 2, FIGS. 9A-D, and FIGS. 12A-15.

The imaging data of the target subject refers to data generated by the radiation rays emitted from the target subject, passing through collimation units of the collimator, and collected by the detector. For example, the imaging device collects the imaging data of the target subject in the at least one scan through the detector. The processing device 140 acquires the imaging data from the imaging device through the network 120.

The imaging data may comprise first imaging data corresponding to the first collimation units and second imaging data corresponding to the second collimation units. The first imaging data refers to imaging data collected after the radiation rays passing through the first collimation units are received by the detector modules. The first FOV corresponding to the first collimation units is a large field of view with a low resolution. Therefore, the first imaging data is data with a low resolution but a large field of view. The second imaging data refers to imaging data collected after the radiation rays passing through the second collimation units are received by the detector modules. The second FOV corresponding to the second collimation units is a small field of view with a high resolution. Therefore, the second imaging data is data with a high resolution but a small field of view.

In some embodiments, the imaging device includes a plurality of collimator rings and a plurality of detector rings, and the at least one scan may include only a single scan. In the single scan, the first collimation units of a portion of the plurality of collimator rings are in the active state, and the second collimation units of another portion of the plurality of collimator rings are in the active state. Only by the single scan, first imaging data corresponding to the first collimation units and second imaging data corresponding to the second collimation units can be acquired. For example, the plurality of collimator rings include the collimator rings 920-1 and 920-2 shown in FIG. 15. The fifth collimation units in the collimator ring 920-1 may serve as the first collimation units. The data collected by the detector ring 910-1 corresponding to the collimator ring 920-1 is first imaging data. The third collimation units and the fourth collimation units in the collimator ring 920-2 may serve as the second collimation units. The data collected by the detector ring 910-2 corresponding to the collimator ring 920-2 is the second imaging data.

In some embodiments, the imaging device includes one or more collimator rings and one or more detector rings, and the at least one scan includes a first scan and a second scan. During the first scan, the first collimation units of each of the one or more collimator rings are in the active state. The data collected by the one or more detector rings during the first scan is first imaging data. During the second scan, the second collimation units of each of the one or more collimator rings are in the active state. The data collected by the one or more detector rings during the second scan is second imaging data. In some embodiments, the durations of the first scan and the second scan are the same. In some embodiments, considering that first imaging data collected by the first scan has a low resolution, and to improve the overall resolution of the data, a first duration of the first scan is shorter than a second duration of the second scan.

In 1620, the processing device 140 generates a target image of the target subject based on first imaging data and second imaging data.

The target image is a mixed-resolution image, which has a high resolution in a corresponding portion of the second FOV (i.e., the small field of view), and a low resolution in other portions.

In some embodiments, operation 1620 includes operations 1621 and 1622. The processing device 140 may obtain a low resolution complete image based on first imaging data, and then improve the resolution of local regions in the complete image based on second imaging data.

In 1621, the processing device 140 may reconstruct the first imaging data to obtain an initial image. The initial image is an image with a low-resolution and a large field of view, which corresponds to the first FOV. A reconstruction algorithm may include but is not limited to a filtered back projection algorithm, a Maximum Likelihood Expectation Maximization (MLEM) manner, a Least Squares (LS) manner, an ordered subset expectation maximization (OSEM) algorithm, or the like.

In 1622, the processing device 140 may iteratively update the initial image based on the second imaging data to generate the target image. The iterative updating of the initial image based on the second imaging data may also be implemented based on the reconstruction algorithm described above.

In some embodiments, the processing device 140 may determine correction data based on the first imaging data. The correction data may be an attenuation coefficient map of the target subject. For example, the processing device 140 may determine tissue types of different portions of the target subject based on the first imaging data, and determine corresponding attenuation coefficients based on the tissue types. The processing device 140 may iteratively update the initial image based on the correction data and the second imaging data to generate the target image. Merely by way of example, the processing device 140 performs attenuation correction on the second imaging data based on the correction data, then reconstructs the corrected second imaging data to obtain an updated image, the updated image is an image with a high-resolution and a small FOV, then performs registration and fusion on the updated image and the initial image to generate the target image. In some other embodiments, the processing device 140 may reconstruct an image with a high-resolution and a small FOV based on the second imaging data, and then perform registration and fusion on the image and the initial image to generate the target image.

In some embodiments of the present disclosure, obtaining the initial image based on the first imaging data corresponding to a large FOV and a low resolution may ensure that the target image covers a large FOV range, and iteratively updating the initial image based on the second imaging data corresponding to a small FOV and a higher resolution may ensure that local details of the target image include more information, thereby improving image quality.

In some embodiments, operation 1620 includes operation 1623.

In 1623, the processing device 140 performs a joint reconstruction on the first imaging data and the second imaging data to generate the target image.

In some embodiments, the joint reconstruction may also be implemented based on the reconstruction algorithm described above. In some embodiments, the processing device 140 performs the joint reconstruction on the first imaging data and the second imaging data based on the first duration of the first scan and the second duration of the second scan to generate the target image. The processing device 140 may assign weights to the first imaging data and the second imaging data based on the first duration and the second duration, and then perform the joint reconstruction by a preset reconstruction algorithm. For example, when the first duration is shorter than the second duration, the first duration corresponds to a lower weight. Merely by way of example, the joint reconstruction on the first imaging data and the second imaging data may be implemented by formula (1):

$${x}^{n + 1} = \frac{{x}^{n}}{{\sum }_{i = 0}^{1} {w}_{i} \cdot {P}_{i}^{T} \cdot 1} {\sum }_{i = 0}^{1} {\sum }_{i = 0}^{1} {w}_{i} \cdot {P}_{i}^{T} \frac{{y}_{i}}{{w}_{i} \cdot {P}_{i} x + {s}_{i}} \qquad ( 1 )$$

xⁿ⁺¹ and xⁿ are respectively the reconstructed images corresponding to the (n+1)-th and n-th iterations, when n+1 is the last iteration, xⁿ⁺¹ corresponds to the target image; i=0 and i=1 correspond respectively to the first scan and the second scan; wᵢ represents the weight of the imaging data collected by each scan, which satisfies w₀+w₁=1; Pᵢ is a system matrix, which characterizes a mapping relationship from an image space to a projection space; yᵢ is the imaging data collected by each scan (the first imaging data or the second imaging data); sᵢ is a scattering estimate value corresponding to each scan, used for compensating for interference of scattering events on projection data. The weight of the imaging data may be determined based on a scan duration corresponding thereto. Taking the first imaging data as an example, w₀=the first duration/(the first duration+the second duration).

In some embodiments of the present disclosure, by weighted combining the first imaging data and the second imaging data based on the first duration and the second duration and introducing scattering correction, it is possible to effectively utilize complementary information of different FOVs, improve the spatial resolution and quantitative accuracy of the reconstructed image, and adapt to a difference in two types of aperture sensitivities, thereby achieving an optimized compromise between system sensitivity and resolution.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an subject oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (Saas).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lies in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate a certain variation (e.g., ±1%, ±5%, ±10%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value.”

1. An imaging system, comprising an imaging device, wherein:
the imaging device comprises a plurality of detector rings and a plurality of collimator rings,
the plurality of detector rings are arranged side by side along an axial direction of the imaging device, each of the plurality of detector rings comprises a plurality of detection modules arranged at intervals along a circumferential direction of the imaging device, and the detection modules of adjacent detector rings are staggered along the circumferential direction, and
each of the plurality of collimator rings is nested within a channel formed by one of the plurality of detector rings.

2. The imaging system of claim 1, wherein for each of the plurality of collimator rings, the collimator ring is rotatable relative to the corresponding detector ring about the axial direction of the imaging device.

3. The imaging system of claim 2, wherein for each of the plurality of collimator rings,
the collimator ring comprises collimation units of a plurality of types, different types of collimation units having different structures,
a rotation of the collimator ring causes one type of the collimation units to be in an active state, and one or more other types of the collimation units to be in an inactive state.

4. The imaging system of claim 3, wherein the structures of the collimation units in the active state among the plurality of collimator rings are different.

5. The imaging system of claim 3, wherein the structures of the collimation units in the active state among the plurality of collimator rings are the same.

6. The imaging system of claim 3, wherein for each of the plurality of collimator rings, the collimation units comprise first collimation units of a first type and second collimation units of a second type,
a first field of view (FOV) is achieved when the rotation of the collimator ring causes the first collimation units to be in the active state,
a second FOV is achieved when the rotation of the collimator ring causes the second collimation units to be in the active state, and
the first FOV is larger than the second FOV.

7. The imaging system of claim 6, further comprising a processing device configured for:
acquiring imaging data collected by the imaging device in at least one scan of a target subject, the imaging data comprising first imaging data corresponding to the first collimation units and second imaging data corresponding to the second collimation units; and
generating a target image of the target subject based on the first imaging data and the second imaging data.

8. The imaging system of claim 7, wherein the generating the target image of the target subject based on the first imaging data and the second imaging data comprises:
reconstructing the first imaging data to obtain an initial image; and
iteratively updating the initial image based on the second imaging data to generate the target image.

9. The imaging system of claim 7, wherein:
the at least one scan comprises a first scan and a second scan, the first collimation units of each of the plurality of collimator rings are in the active state during the first scan, and the second collimation units of each of the plurality of collimator rings are in the active state during the second scan,
a first duration of the first scan is shorter than a second duration of the second scan, and
the target image is generated by performing a joint reconstruction on the first imaging data and the second imaging data based on the first duration and the second duration.

10. The imaging system of claim 3, further comprising a processing device configured for:
determining, based on information relating to a target subject to be scanned, the structure of the collimation units that are in the active state in each of the plurality of collimator rings.

11. The imaging system of claim 1, wherein the plurality of detector rings include a target detector ring, the plurality of collimator rings include a target collimator ring, and the target collimator ring is nested within a channel formed by the target detector ring,
the target collimator ring includes third collimation units and fourth collimation units,
first projections of the third collimation units along a radial direction of the imaging device cover at least a portion of the plurality of detection modules of the target detector ring and are independent from each other, and
second projections of the fourth collimation units along the radial direction of the imaging device cover at least a portion of the plurality of detection modules of the target detector ring and at least partially overlap with each other.

12. The imaging system of claim 11, wherein the plurality of detection modules of the target detector ring include first detection modules and second detection modules,
the first projections of the third collimation units along the radial direction of the imaging device cover the first detection modules and are independent from each other,
the second projections of the fourth collimation units along the radial direction of the imaging device cover the second detection modules and at least partially overlap with each other.

13. The imaging system of claim 12, wherein a size of the first detection modules is greater than or equal to a size of the second detection modules.

14. The imaging system of claim 12, wherein a number of the first detection modules is greater than or equal to a number of the second detection modules.

15. The imaging system of claim 11, wherein for each of the fourth collimation units, an area of an overlapping region of its corresponding second projection with second projections of other fourth collimation units accounts for no more than 50% of a total area of the second projection.

16. The imaging system of claim 11, wherein the target collimator ring includes first collimation modules and second collimation modules, the third collimation units being disposed on the first collimation modules, and the fourth collimation units being disposed on the second collimation modules;
a size of the first collimation modules is greater than a size of the second collimation modules, and
the first collimation modules and the second collimation modules are at a first distance from a central axis of the imaging device.

17. The imaging system of claim 16, wherein the target collimator ring further includes third collimation modules, the third collimation modules being at a second distance from the central axis of the imaging device, the second distance being greater than the first distance,
fifth collimation units are disposed on the third collimation modules,
the fifth collimation units correspond to a first FOV, and the third collimation units and the fourth collimation units correspond to a second FOV, the first FOV being greater than the second FOV,
the target collimator ring is rotatable relative to the target detector ring about the central axis of the imaging device to switch the target detector ring between the first FOV and the second FOV.

18. An imaging method, comprising:
acquiring imaging data collected by the imaging device of claim 6 in at least one scan of a target subject, the imaging data including first imaging data corresponding to the first collimation units and second imaging data corresponding to the second collimation units; and
generating a target image of the target subject based on the first imaging data and the second imaging data.

19. The imaging method of claim 18, wherein the generating a target image of the target subject based on the first imaging data and the second imaging data comprises:
reconstructing the first imaging data to obtain an initial image; and
iteratively updating the initial image based on the second imaging data to generate the target image.

20. The imaging method of claim 18, wherein the at least one scan comprises a first scan and a second scan, the first collimation units of each of the plurality of collimator rings being in the active state during the first scan, and the second collimation units of each of the plurality of collimator rings being in the active state during the second scan,
a first duration of the first scan is shorter than a second duration of the second scan, and
the target image is generated by performing a joint reconstruction on the first imaging data and the second imaging data based on the first duration and the second duration.

21-27. (canceled)
